(19) United States
(12) Patent Application Publication
(10) Pub. No.: US 2014/0193910 A1
(43) Pub. Date: Jul. 10, 2014

(54) METHODS FOR PRODUCING PANCREATIC PRECURSOR CELLS AND USES THEREOF

(71) Applicants: James L. Sherley, Boston, MA (US); Jean-Francois Pare, Watertown, MA (US)

(72) Inventors: James L. Sherley, Boston, MA (US); Jean-Francois Pare, Watertown, MA (US)

(21) Appl. No.: 14/125,665

(22) PCT Filed: Jun. 15, 2012
(86) PCT No.: PCT/US12/42644

§ 371 Date: Mar. 17, 2014

Related U.S. Application Data
(60) Provisional application No. 61/497,257, filed on Jun. 15, 2011.

(51) Int. Cl. C12N 5/071

(52) U.S. Cl. CPC C12N 5/0678
USPC 435/377

(57) ABSTRACT

The present invention is directed to methods for readily propagating somatic pancreatic precursor cells. The methods comprise isolating cells from intact pancreatic samples and enhancing guanine nucleotide (GNP) biosynthesis in cultures comprising these cells, thereby expanding guanine nucleotide pools. This in turn conditionally suppresses asymmetric cell kinetics in the cells, thereby generating pancreatic precursor cells.

### CROSS-REFERENCE TO RELATED APPLICATIONS

This application claims benefit under 35 U.S.C. §119(e) of U.S. Provisional Patent Application Ser. No. 61/497,257 filed on 15 Jun. 2011, the contents of which are incorporated herein by reference in its entirety.

### GOVERNMENT SUPPORT

This invention was made with Government support under NIH Director's Pioneer Award 5DP1OD000805 awarded by the National Institutes of Health (NIH). The Government has certain rights in the invention.

### FIELD OF THE INVENTION

The present application is directed to the ex vivo expansion of pancreatic precursor cells and to their use in cell replacement therapies, including transplantation therapies and tissue engineering applications, as well as for research and drug evaluation applications. Preferably pancreatic precursor cells from human tissue are used.

### BACKGROUND OF THE INVENTION

Stem cells have the ability to differentiate into a variety of cells and tissues. Thus, considerable attention has focused on stem cells and their uses in a multitude of applications, including tissue engineering, tissue regeneration, and gene therapy. Stem cells have been isolated from both embryonic and adult tissues. Somatic stem cells that are derived from adult tissue still have the ability to renew adult tissues (Fuchs and Segre, 2000). Thus, in light of the ongoing controversies surrounding the use of embryonic stem cells, the use of somatic stem cells or somatic precursor cells are a particularly attractive alternative.

The presence of stem cells in somatic tissues has been well established using functional tissue cell transplantation assays (Reisner et al., 1978). However, isolation and propagation of somatic stem cells has proven difficult. Methods to isolate and expand stem cells from somatic tissue, particularly without significant differentiation, are highly desirable. There have been some questions raised regarding how multi-potent adult stem cells are related to embryonic stem cells. Thus, it is important to be able to obtain and cultivate many different types of somatic stem cells.

In particular, the availability of a method for producing pancreatic precursor cells from adult tissues would greatly contribute to cell replacement therapies and tissue engineering. For example, transplantable pancreatic precursor cells with the ability to produce glucose-responsive β-cells could address the shortage of donor islets.

There has been considerable difficulty encountered in obtaining pancreatic stem cells or precursor cells that can be propagated and cultured ex vivo. One factor is the predominant way somatic stem cells divide is by asymmetric cell kinetics. During asymmetric kinetics, one daughter cell divides with the same kinetics as its stem cell parent, while the second daughter gives rise to a differentiating non-dividing cell lineage cell. The second daughter may differentiate immediately; or, depending on the tissue, it may undergo a finite number of successive symmetric divisions to give rise to a larger pool of differentiating cells.

Such asymmetric cell kinetics are a major obstacle to somatic cell expansion in vitro (Merok and Sherley, 2001; Rambhatla et al., 2001; Sherley, 2002). In culture, continued asymmetric cell kinetics results in dilution and loss of an initial relatively fixed number of stem cells by the accumulation of much greater numbers of their terminally differentiating progeny. If a sample includes both exponentially growing cells as well as somatic stem cells, the growth of the exponentially growing cells will rapidly overwhelm the somatic stem cells, leading to their dilution. Even in instances where it is possible to select for relatively purer populations, for example by cell sorting, asymmetric cell kinetics prevent expansion.

Another factor in obtaining pancreatic stem cells or precursor cells that can be propagated and cultured ex vivo is that most methods used for generating pancreatic stem cells or pancreatic precursor cells comprise a step of purifying, enriching for, or separating pancreatic islet cells prior to cell culture. This limits the number of starting cells that can be used in cultures.

### SUMMARY OF THE INVENTION

Provided herein are novel methods for readily propagating pancreatic stem cells or pancreatic precursor cells that produce insulin, glucagon, or a combination thereof, for use in stem cell transplantation therapies and other applications. Type I diabetes (T1D) is a devastating disease that results from autoimmune destruction of pancreatic β-cells. Transplantation of functional islets is the most reliable way to restore normal glucose homeostasis in T1D patients, but the scarcity of donor islets limits this treatment strategy. In vitro expansion of transplantable pancreatic stem cells with the ability to produce glucose-responsive β-cells could address the shortage of donor islets.

As described herein, we determined that proteolytic release of total cells from intact human pancreas samples, using at least two “digestion steps,” is critical for the production and propagation of human pancreatic progenitor cells using the methods described herein. Following the proteolytic release, the cells are incubated in culture medium supplemented with specific guanine ribonucleotide precursors (GrNPs). Specific purine precursors are used to expand cellular guanine ribonucleotide (rGNP) pools. The GrNPs promote the shift of the human pancreatic stem cells, present in the total cell mixture produced by the proteolytic release of cells from intact human pancreas samples, from their homeostatic asymmetric self-renewal, which keeps their numbers constant, to symmetric self-renewal, which promotes their exponential multiplication. Since the shift to symmetric self-renewal is reversible, expanded cells can be returned to asymmetric self-renewal and homeostatic renewal of functional tissue cells by withdrawal of the suppressing purine precursors, which occurs upon transplantation.

As described herein, supplementation of culture media with purine precursors promotes the growth and increases the clonogenicity of human pancreatic stem cell or pancreatic precursor cell strains obtained from intact human pancreatic samples. Human C-peptide was detected in the blood of mice injected intraperitoneally with undifferentiated human pancreatic stem cells demonstrating that pancreatic precursor cells, such as pancreatic stem cells, produced using the methods described herein can mature into insulin-secreting cells. Further, as shown herein, the derived cells express the pancreatic endocrine progenitor Ngn3 and the expanded human pancreatic stem cells or pancreatic precursor cells form islet-like clusters under differentiation conditions in vitro. Further, as demonstrated herein, the majority of a population of pancreatic cells, including expanded from pancreatic precursor cells, produced using the methods described herein can co-express the hormones insulin and glucagon within the same cells, suggesting that they represent immature islets.

Accordingly, in some aspects, provided herein are methods for culturing and expanding somatic pancreatic precursor cells in vitro. These methods comprise: a) twice digesting (or digesting at least two times) a population of pancreatic cells isolated from an intact pancreatic tissue sample obtained from a mammal, where the population of pancreatic cells comprises pancreatic alpha cells, pancreatic beta cells, pancreatic delta cells, pancreatic polypeptide (PP) cells, acinar cells, ductal cells, pancreatic precursor cells, mesenchymal cells, fibroblasts, or a mixture or combination thereof, followed by b) culturing the population of pancreatic cells in a culture medium that permits cell growth under conditions, and for a time sufficient, to permit cell growth, such that the culture medium comprises at least 50 μm of a guanine nucleotide precursor selected from xanthosine, xanthine, or hypoxanthine, or an analogue or derivative thereof, where the guanine nucleotide precursor, analogue or derivative thereof suppresses asymmetric cell kinetics, thereby allowing exponential growth and expansion of the pancreatic precursor cells.

In some embodiments of these methods and all such methods described herein, the twice digesting step does not comprise or is not followed by purifying, enriching for, or separating pancreatic alpha cells, pancreatic beta cells, or any combination thereof, prior to the step of culturing.

In some embodiments of these methods and all such methods described herein, the guanine nucleotide precursor is xanthosine, hypoxanthine, or a combination thereof. In some embodiments of these methods, the guanine nucleotide precursor does not comprise xanthine.

In other embodiments of these methods and all such methods described herein, the guanine nucleotide precursor is xanthosine, xanthine, or a combination thereof. In some embodiments of these methods and all such methods described herein, the guanine nucleotide precursor does not comprise hypoxanthine.

In some embodiments of these methods and all such methods described herein, the guanine nucleotide precursor is present in an amount of at least 50-200 μM. In some embodiments of these methods and all such methods described herein, the guanine nucleotide precursor is present in an amount of at least 200 μM. In some such embodiments of these methods, the guanine nucleotide precursor is present in an amount of 200 μM-2 mM. In some such embodiments of these methods, the guanine nucleotide precursor is present in an amount of 500 μM-2 mM.

In some embodiments of these methods and all such methods described herein, the pancreatic precursor cells can differentiate into a pancreatic alpha cell or a pancreatic beta cell.

In some embodiments of these methods and all such methods described herein, the pancreatic precursor cells produce or secrete insulin, glucagon, or any combination thereof.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of elements that do not materially affect the basic and novel or functional characteristic(s) of that embodiment of the invention.

The term “consisting of refers to compositions, methods, and respective components thereof as described herein, which are exclusive of any element not recited in that description of the embodiment.

As used in this specification and the appended claims, the singular forms “a,” “an,” and the include plural references unless the context clearly dictates otherwise. Thus for example, references to the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol.152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

### BRIEF DESCRIPTION OF THE DRAWINGS

FIGS. 1A-1C demonstrate exemplary islet differentiation properties of an expanded clonal human pancreatic stem cell strain produced using an embodiment of the methods described herein. A clonal strain, derived, using one embodiment of the methods described herein, in CMRL-1066-10% DFBS supplemented with 1 mM Xs, was evaluated by in situ indirect immunofluorescence (ISIF) for expression of the indicated pancreatic phenotype markers after 4 days of culture in Xs-free islet differentiation medium. Islet-like cell clusters were sectioned for analysis. FIG. 1A shows co-expression of glucagon (gluc) and insulin (ins). Arrows indicate examples of cells that express only glucagon or only insulin. FIG. 1B shows magnified views of single-positive cells indicated in FIG. 1A. FIG. 1C shows co-expression of glucagon and neurogenin-3 (Ngn-3). Phase, corresponding phase micrographs. DAPI, corresponding nuclear DNA fluorescence. (Note: ISIF analyses without primary marker-specific antibodies showed no detectable secondary antibody fluorescence under the conditions shown.)

FIG. 2 depicts a schematic of adult stem cell kinetics and the SACK method. When explanted to culture, adult stem cells keep their basic asymmetric cell kinetics, which limits their ex vivo expansion and dilutes them in an increasing pool of transit-amplifying and differentiated cells. In the suppression asymmetric cell kinetics (SACK) method, exogenous purine precursors (SACK agents) are supplemented to the culture medium to reversibly induce symmetric cell kinetics in the adult stem cells., which leads to their expansion. Withdrawal of the SACK agent(s) results in the reacquisition of asymmetric cell kinetics and differentiation of the stem cell progeny when in the appropriate microenvironment.

FIG. 3 depicts a metabolic pathway regulating stem cell kinetics. Asymmetric cell kinetics require down-regulation of IMPDH by p53. In the SACK method, hypoxanthine (Hx), xanthine (Xn), and xanthosine (Xs) are exogenous purine precursors termed herein as “SACK agents” and are used to expand the cellular guanine ribonucleotide (rGNP) pools. They increase the cellular levels of rGNPs independently of p53 activity. As a result, adult stem cells, which normally divide assymmetrically, acquire symmetric cell kinetics and expand. GMP, guanosine-5′-monophosphate; HGPRT, hypoxanthine-guanine phosphoribosyltransferase; IMP, inosine-5′-monophosphate; IMPDH, IMP dehydrogenase; Nsk, nucleoside kinase; UA, uric acid; XO, xanthine oxidase.

FIG. 4 depicts a schematic of a derivation of human pancreatic stem cell strains. Isolation of stem cell strains from human pancreatic tissue was performed as described herein. Briefly, following mincing, human pancreatic tissue (provided by National Disease Research Interchange) was digested in 1.0 mg/mL LIBERASE™ HI for 20 minutes at 37° C. Cells and clusters released were pelleted and plated equally in the indicated culture conditions. Undigested tissue was submitted to one more identical digestion and plating, and tissue still undigested was also plated in the indicated conditions. Two weeks after plating, attached cells were released by trypsinization and replated in the same conditions. Clones were picked and expanded four weeks after replating. Cells left on the plates after harvesting the clones were pooled and used for growth curves. All culture conditions contained 10% dialysed fetal bovine serum.

FIG. 5 demonstrates that purines promote growth and clonogenicity. Growth properties of human pancreatic stem cell strains. Four weeks after replating, clones were picked and expanded. Cells left after clonal isolations from the CMRL-1066 conditions were pooled and grown. They were trypsinized and counted on a regular basis, and population doublings were calculated accordingly. The number of clones detected for each condition is represented on the top two histograms. The bottom histogram represents the percentage of harvested clones that continued their expansion after isolation.

FIG. 6 demonstrates secretion of human C-peptide after transplantation of the pancreatic stem cells produced using an embodiment of the methods described herein. Detection of human C-peptide in the serum of mice transplanted with undifferentiated human pancreatic stem cell strains is shown. Pools of human pancreatic stem cells from the CMRL-1066 conditions described herein were used for transplantation in immunodeficient mice. For each pool, two mice were injected intraperitoneally with 10⁶ cells each. Six months later, mice were anesthesized and their blood was drained by cardiac puncture. Serum levels of human C-peptide were measured using a human C-peptide-specific ELISA kit (Millipore).

FIG. 7 demonstrates expression of endocrine markers by pancreatic stem cells produced by an embodiment of the methods described herein. Detection of pancreatic transcription factors expression by immunofluorescence is shown. Under proliferating conditions, human pancreatic stem cell strains express the endocrine progenitor marker Neurogenin-3 (Ngn3) in their nuclei (left panel). The a-cell marker Arx is detected in the nuclei of cells grown under the control conditions only (right panel).

FIGS. 8A-8B demonstrate formation of islet-like clusters in vitro from pancreatic stem cells produced by an embodiment of the methods described herein. FIG. 8A shows formation of islet-like clusters from the human pancreatic stem cells strains. Actively growing cells were released by trypsinization, washed thrice, resuspended in differentiation medium, and transferred in ultra-low attachment plates (Costar). Differentiation medium consists of CMRL-1066 supplemented with 1% fatty acid-free bovine serum albumin (Sigma), 2 mM L-glutamine, and 1× insulin-transferrin-selenium A (Invitrogen). FIG. 8B shows islets that were harvested after four days of differentiation, concentrated, and frozen in OCT (Tissue-Tek). 10 mm cryosections were mounted on glass slides and protein expression was analyzed by immunofluorescence. The right panel indicates co-expression of insulin and glucagon within the same cells. Scale bar=50 μm.

FIG. 9 demonstrates detection of expression of an endocrine progenitor cell marker Neurogenin 3 (Ngn3) in differentiated, SACK-expanded, human pancreatic stem cell strain HuPan 20. Indirect in situ immunofluorescence analyses were performed with specific anti-Ngn3 antibodies against sections of islet-like clusters produced by differentiation of HuPan cells in SACK-free differentiation medium for 6 days. Scale bar=20 microns.

FIG. 10 demonstrates that differentiated cell clusters produced from expanded human pancreatic stem cells using an embodiment of the methods described herein have expression properties of human islets. Clusters produced in the presence of glucose were sectioned and examined by indirect in situ immunofluorescence with specific antibodies for human insulin (beta-cell biomarker), C-peptide (insulin secretion byproduct), and glucagon (alpha-cell biomarker). Control, no specific antibody included. DAPI, indicator for nuclear DNA. Scale bar=100 microns.

### DETAILED DESCRIPTION OF THE INVENTION

As described herein, we have discovered methods for the production and expansion of human pancreatic stem cells that are precursors for functional human pancreatic islet cells for use in transplantation therapies for disorders such as, for example, Type I diabetes (T1D). The methods provided herein can be used to derive both polyclonal and clonal human pancreatic stem cell strains that can be used in transplantation therapies, including homologous cell transplantation therapies, as well as research and drug evaluation applications. Pancreatic precursor cell strains generated using the methods described herein can be induced to make differentiated progeny cells with both beta-cell function, as defined by insulin production, and alpha-cell function, as defined by glucagon production. The ability to make both types of islet cells is a unique feature and special advantage of the methods described herein. For example, providing both islet functions after transplantation can achieve restoration of normal blood glucose regulation in diabetic patients.

We investigated whether the previously described method of “suppression of asymmetric cell kinetics” or SACK (Lee et al., 2003; Pare and Sherley, 2006; Sherley et al., 2010; Sherley and King, 2010; Sherley and Panchalingam, 2010) for ex vivo expansion of diverse tissue stem cells of various origins can be used to expand adult human pancreatic stem cells with properties required for T1D cell transplantation therapy. The methods described herein, in part, shift pancreatic stem cells from asymmetric cell kinetics to symmetric cell kinetics, which promote exponential expansion of adult stem cells in culture. Symmetric stem cell kinetics are characterized by divisions that produce two stem cells and no differentiating cells. This shift in kinetics symmetry is referred to herein as “suppression of asymmetric cell kinetics” or “SACK.” Methods based, in part, on suppression of asymmetric cell kinetics, as described herein, comprise enhancing guanine nucleotide (GNP) biosynthesis, thereby expanding guanine nucleotide pools. This in turn conditionally suppresses the asymmetric cell kinetics exhibited by, for example, pancreatic precursor cells. One preferred embodiment of the methods described herein enhances guanine nucleotide biosynthesis to bypass or override normal inosine-5′-monophosphate dehydrogenase (IMPDH) regulation. IMPDH catalyzes the conversion of inosine-5′ monophosphate (IMP) to xanthosine monophosphate (XMP) for guanine nucleotide biosynthesis. This step can be bypassed or overridden by providing a guanine nucleotide precursor (rGNPr) such as xanthosine or hypoxanthine, respectively, as described herein. The next metabolite in the GNP pathway is guanine monophosphate (GMP), which in turn is metabolized to the cellular guanine nucleotides.

As described herein, we evaluated the purine nucleoside xanthosine and the purine base xanthine for their ability to promote the expansion of pancreatic precursor cells, which are undifferentiated cells that can subsequently differentiate and include pancreatic stem cells, from adult human cadaveric intact pancreas samples. Supplementation with these SACK agents increased the frequency of primary cell colony growth approximately 50%, while the clonogenicity of initial cell colonies was improved 3-fold. The produced cell strains were identified as pancreatic precursor cell strains by their ability, under culture conditions of GrNP supplementation, to multiply actively and for extended periods (at least 20 populations doublings) without expression of markers of islet cell differentiation.

Importantly, when cultured in SACK-free medium or GrNP-free media under conditions known to induce islet cell differentiation, these pancreatic precursor cells form islet-like clusters and produce progeny cells with properties of both mature pancreatic beta-cells and alpha cells. In addition, under conditions of active multiplication or differentiated cell production, the expanded pancreatic precursor cells produced using the methods described herein express the transcription factor neurogenin-3 (Ngn-3), a phenotypic marker for pancreatic endocrine precursor cells. Further, cells in the clusters co-express human glucagon and human insulin, indicating the production of pancreatic precursor cells with potential and ability to establish subsequent lineages with either α-cell or β-cell phenotype, respectively. Moreover, cluster cells also expressed human C-peptide, an indicator of complete insulin production capability. In addition, in a cell transplantation study with the SACK-derived cells, human C-peptide was detected in the blood of injected immunodeficient mice at levels comparable to fasting human blood insulin levels (0.11 ng/ml). Further, it was found that SACK-expanded mouse pancreatic stem cells home specifically to the pancreas after injection into the peritoneal cavity of congenic mice. Although the exact anatomical route of passage has not been determined, without wishing to be bound or limited by theory, it is likely to be via ingress into subdiaphragmatic lymphatics, passage through the thoracic duct, return to the venous circulation, and retrograde diapedesis through pancreatic post-capillary venules.

### Pancreatic Precursor Cells: Isolation and Culture Thereof

Surprisingly, we determined that using a population of only intact islet cells with the SACK methods to expand the number of undifferentiated pancreatic precursor cells was ineffective, and that dissociating the entire pancreas or using a an intact pancreatic tissue sample, instead of starting with isolated islets, unexpectedly and significantly enhanced the number of initial cell colonies produced, as well as the ability to propagate initial colonies as clonal strains.

Pancreatic precursor cells, such as pancreatic stem cells, described herein can be isolated from intact pancreatic tissue of an adult mammal, preferably a human. As used herein, “intact pancreatic tissue” refers to any pancreatic tissue sample obtained from an adult mammal that is not manipulated or altered in such a way as to, for example, enrich for or purify a specific population of pancreatic cells. Accordingly, an intact pancreatic tissue refers to a tissue or sample comprising more than just pancreatic islet cells (i.e., alpha cells, beta cells, delta cells, and PP cells), and including one or more other cell types found in an intact pancreas, including, for example, acinar cells, ductal cells, and any pancreatic progenitor or precursor cells thereof, mesenchymal cells, fibroblasts, and any other cells present in the pancreatic connective tissue, as well as other cells (e.g., endothelial cells, neuronal cells, and progenitor or precursor cells that are not differentiated or not fully differentiated or yet to be differentiated), or any mixture or combination thereof. For example, a pancreatic biopsy sample obtained from a subject is an intact pancreatic tissue sample, as the term is used herein.

Such intact pancreatic tissue samples can be used for isolating populations of pancreatic cells for use in the methods described herein. In some embodiments of the methods described herein, an intact pancreatic tissue sample is at least 0.5 cm in one dimension, or preferably, at least 1 cm in one dimension, at least 2 cm in one dimension, at least 3 cm in one dimension, at least 4 cm in one dimension, at least 5 cm in one dimension, at least 6 cm in one dimension, at least 7 cm in one dimension, at least 8 cm in one dimension, at least 9 cm in one dimension, at least 10 cm in one dimension, at least 15 cm in one dimension, at least 20 cm in one dimension, or more.

The terms “isolate” and “methods of isolation,” as used herein, refer to any process whereby a cell or population of cells, such as a population of pancreatic cells, is removed from a subject or sample in which it was originally found, or a descendant of such a cell or cells. The term “isolated population,” as used herein, refers to a population of cells that has been removed and separated from a biological sample, or a mixed or heterogeneous population of cells found in such a sample. Such a mixed population includes, for example, a population of pancreatic cells obtained from an intact pancreatic sample, or a cell suspension obtained from such a pancreatic tissue sample. In some embodiments of the methods described herein, an isolated cell or cell population, such as a population of pancreatic cells obtained from an intact pancreatic tissue, is further cultured in vitro or ex vivo, e.g., in the presence of guanine nucleotide precursors, growth factors or cytokines, to further expand the number of cells in the pancreatic cell population. Such culture can be performed using any method known to one of skill in the art, for example, as described in the Examples section. In some embodiments of the methods described herein, the pancreatic precursor cell populations obtained using the methods disclosed herein are later administered to a second subject, or, in some embodiments, reintroduced into the subject from which the cell population was originally isolated (e.g., allogeneic or heterologous transplantation vs. autologous or homologous administration).

The term “population of pancreatic cells,” as used herein, refers to a preparation of cells obtained or isolated from an intact pancreatic tissue, and includes both endocrine and exocrine pancreatic tissue cells, as well as cell lines derived therefrom. The endocrine pancreas is composed of hormone producing cells arranged in clusters known as islets of Langerhans or islets. Of the four main types of cells that form the islets (termed herein as “islet cells”), “alpha cells” or “α cells” refer to islet cells that produce glucagons; “beta cells” or “β cells” refer to islet cells that produce insulin; “delta cells” or “δcells” refer to islet cells that produce somatostatin; and “pancreatic polypeptide cells” or “PP cells” refer to islet cells that produce pancreatic polypeptide (PP). The exocrine pancreas includes the pancreatic acini and the pancreatic duct. “Pancreatic acinar cells,” as used herein, refer to those pancreatic cells that synthesize a range of digestive enzymes. “Ductal cells,” as used herein, refer to those pancreatic cells that secrete bicarbonate ions and water in response to hormones secreted from the gastrointestinal tract. Therefore, the term “pancreatic cells,” as used herein, includes cells found in an intact pancreas, including alpha cells, beta cells, delta cells, PP cells, acinar cells, ductal cells, and any pancreatic progenitor or precursor cells thereof, mesenchymal cells, fibroblasts, and any other cells present in the pancreatic connective tissue, as well as other cells (e.g., endothelial cells, neuronal cells, and progenitor or precursor cells that are not differentiated or not fully differentiated or yet to be differentiated), or any mixture or combination thereof.

Accordingly, the terms “islet,” “islet cells,” or “islets,” as used herein, include the constituent cell types within the islet of Langerhans, including alpha, beta, delta, pancreatic polypeptide cells, and epsilon cells, intact islets, islet fragments, or any combinations thereof.

The terms “progenitor cell” or “somatic stem cell,” as used herein refer to an undifferentiated cell that is capable of proliferation and also has the ability to generate differentiated, or differentiable cells. At a minimum, the term “progenitor cell” refers to a pancreatic progenitor cell lineage that is able to produce cells of the pancreas and encompasses e.g., multipotent, pluripotent and/or totipotent cells. In some embodiments, the term “progenitor cell” also encompasses a cell which is sometimes referred to in the art as a “stem cell”. In some embodiments, the term “progenitor cell” refers to a generalized mother cell whose descendants (progeny) specialize, often in different directions, by differentiation, e.g., by acquiring completely individual characters, as occurs in progressive diversification of embryonic cells and tissues. The term “progenitor cell” also encompasses progenitor cells arising in tissue of a pancreatic intralobular duct and giving rise to such differentiated progeny as, for example, B cell lineages.

Accordingly, the terms “pancreatic precursor cell” or “pancreatic progenitor cell” refer to any cell that can differentiate into a cell of pancreatic lineage, e.g., a cell which can produce a hormone or enzyme normally produced by a pancreatic cell. For instance, a pancreatic precursor cell can be induced to differentiate, at least partially, into an α, β, δ, or φ islet cell, or a cell of exocrine fate. The pancreatic precursor cells produced and expanded using the methods described herein can also be cultured prior to administration to a subject under conditions which promote cell proliferation and differentiation. These conditions include, for example, culturing the cells to allow proliferation and confluence in vitro, at which time the cells can be made to form islet-like aggregates or clusters, and secrete insulin, glucagon, somatostatin, or any combination thereof.

A unique feature of the methods described herein is the generation of a pancreatic precursor cell that produces or secretes both insulin and glucagon. Accordingly, in some embodiments, a pancreatic precursor cell derived using the methods described herein refers to a cell that secretes both insulin and glucagon. Such pancreatic precursor cells can also be referred to herein as “islet precursor cells.”

Pancreatic precursor cells can be isolated from an intact pancreatic tissue sample from an individual in need of pancreatic stem cell therapy, or from another individual, i.e., a “donor” individual. Preferably, the donor individual is an HLA (Human Leukocyte Antigen) matched individual to insure that rejection problems do not occur. Those having ordinary skill in the art can readily identify matched donors using standard techniques and criteria. Other therapies to avoid rejection of foreign cells are known in the art. Pancreatic precursor cells from a matched donor can be administered by any known means, for example, intravenous injection, or injection directly into an appropriate site or tissue, such as the abdominal area or into the pancreas.

A population of pancreatic cells can be obtained or isolated from an intact pancreatic tissue sample, for example, by dissociation of individual cells from the connecting extracellular matrix of the tissue. The tissue sample is removed using a sterile procedure, and the pancreatic cells are dissociated using any method known in the art, including treatment with one or more enzymes, termed herein as “digestion,” such as trypsin, collagenase A, collagenase B, collagenase C, collagenase H, or any combination thereof, as well as any synthetic or commercially available enzymatic preparation, such as, for example, LIBERASE H™, or by using physical methods of dissociation such as mincing or cutting with an instrument. Preferably, at least two digestion steps are used or performed before the cells are cultured in the presence of at least 50 μM of one or more guanine nucleotide precursors.

In some preferred embodiments of the methods described herein, an intact pancreatic sample is obtained under sterile conditions. A population of pancreatic cells is then isolated from the pancreatic sample by first mincing the sample into smaller pieces, and then digesting the minced sample using one or more enzymes, such as, for example, LIBERASE H™, to produce a population of pancreatic cells. In preferred embodiments, the step of digesting is performed at least two times, i.e., twice digested, or, if required or desired, at least three times, at least four times, at least 5 times, or more. The population of pancreatic cells comprising alpha cells, beta cells, delta cells, PP cells, acinar cells, ductal cells, any pancreatic progenitor or precursor cells thereof, mesenchymal cells, and/or fibroblasts, thus obtained is then washed and can then be placed in culture medium for use in subsequent steps of the methods described herein.

In preferred embodiments of the methods described herein, the step of isolating a population of pancreatic cells does not comprise or is not followed by any substantial purification, enrichment, selection, or separation steps. Accordingly, it is preferred that, following the preferably at least two digestion steps of the intact pancreatic sample, the at least twice digested population of pancreatic cells is directly used in the subsequent culturing steps, with no purification or enrichment for specific sub-population(s) of pancreatic cells, for example, of islet cells.

The term “substantially pure” or “purified” with respect to a particular cell population, refers to a population of cells that is at least about 75%, at least about 80%, at least about 85%, at least about 90%, at least about 95%, at least about 98%, or at least about 99% pure, with respect to the cells making up a total cell population. Some embodiments of the aspects described herein further encompass methods to expand a population of pancreatic precursor cells, wherein the expanded population of pancreatic precursor cells is a substantially pure or enriched population of pancreatic precursor cells.

The terms “enriching for or “enriched for” are used interchangeably herein and mean that the yield (fraction) of cells of one type, such as pancreatic precursor cells for use in the methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation. Methods to isolate a substantially pure or enriched population of pancreatic precursor cells available to a skilled artisan include, but are not limited to, immunoselection techniques, such as high-throughput cell sorting using flow cytometric methods, affinity methods with antibodies labeled to magnetic beads, biodegradable beads, non-biodegradable beads, and antibodies panned to surfaces including dishes, and any combination of such methods, as well as separation based on other physical properties (Doevendans et al. (2000) J. Mol. Cell. Cardiol. 32:839-851). Other means of positive selection include drug selection, for instance such as described by Klug et al., involving enrichment of desired cells by density gradient centrifugation. Negative selection can be performed and selecting and removing cells with undesired markers or characteristics, for example fibroblast markers, epithelial cell markers etc.

Any culture medium can be used in the methods of expanding pancreatic precursor cells described herein that is capable of supporting cell growth, including HEM, CMRL, DULBECCO'S MODIFIED EAGLE'S MEDIUM® (DMEM), DMEM F12 MEDIUM®, EAGLE'S MINIMUM ESSENTIAL MEDIUM®, F-12K MEDIUM®, ISCOVE'S MODIFIED DULBECCO'S MEDIUM®, RPMI-1640 MEDIUM®, and serum-free medium for culture and expansion of progenitor cells SFEM®, and the like, containing supplements that are required for cellular metabolism, such as glutamine and other amino acids, vitamins, minerals and useful proteins, such as transferrin and the like. Many media are also available as low-glucose formulations, with or without sodium pyruvate. Medium can also contain antibiotics to prevent contamination with yeast, bacteria and fungi, such as penicillin, streptomycin, gentamicin and the like.

In some embodiments, the culture medium can contain serum derived from bovine, equine, chicken and the like sources. Serum can contain xanthine, hypoxanthine, or other compounds which enhance guanine nucleotide biosynthesis, although generally at levels below the effective concentration to suppress asymmetric cell kinetics. It is understood that sera can be heat-inactivated at 55-65° C. if deemed necessary to inactivate components of the complement cascade.

Thus, in preferred embodiments, a defined, serum-free culture medium is used, as serum contains unknown components (i.e., is undefined). Preferably, if serum is used, it has been dialyzed to remove rGNPrs. A defined culture medium is also preferred if the cells are to be used for cell transplantation purposes. A particularly preferable culture medium is a defined culture medium comprising a mixture of DMEM, F12, and a defined hormone and salt mixture. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for teaching growing cells in serum-free medium or defined medium. As described herein, by including a compound such as a rGNPr, asymmetric cell kinetics are suppressed. Thus, the effect of division by differentiated pancreatic cells, which results in the diluting of the pancreatic precursor cells, is reduced.

Additional supplements also can be used advantageously to supply the cells with the necessary trace elements for optimal growth and expansion, in some embodiments. Such supplements include insulin, transferrin, sodium selenium and combinations thereof. These components can be included in a salt solution such as, but not limited to, HANKS' BALANCED SALT SOLUTION® (HB SS), EARLE'S SALT SOLUTION®, antioxidant supplements, MCDB-201® supplements, phosphate buffered saline (PBS), ascorbic acid and ascorbic acid-2-phosphate, as well as additional amino acids. While many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

A culture medium for use with the methods described herein can be supplemented with one or more proliferation-inducing growth factor(s). As used herein, the term “growth factor” refers to a protein, peptide or other molecule having a growth, proliferative, differentiative, or trophic effect on cells, such as pancreatic precursor cells. Growth factors that can be used include, but are not limited to, any trophic factor that allows a population of pancreatic cells or pancreatic precursor cells to proliferate, including any molecule that binds to a receptor on the surface of the cell to exert a trophic, or growth-inducing effect on the cell. Preferred proliferation-inducing growth factors include, but are not limited to, EGF, amphiregulin, acidic fibroblast growth factor (aFGF or FGF-1), basic fibroblast growth factor (bFGF or FGF-2), transforming growth factor alpha (TGF alpha), and combinations thereof. Commonly used growth factors include, but are not limited to, bone morphogenic protein, basic fibroblast growth factor, platelet-derived growth factor and epidermal growth factor, Stem cell factor, thrombopoietin, Flt3Ligand and 1′-3. Growth factors are typically added to the culture medium at concentrations ranging between about 1 fg/ml to 1 mg/ml. Concentrations between about 1 to about 100 ng/ml are usually sufficient. Simple titration experiments can be easily performed by one of ordinary skill in the art to determine the optimal concentration of a particular growth factor.

In addition to proliferation-inducing growth factors, in some embodiments, other growth factors can be added to the culture medium that influence proliferation and differentiation of the cells including NGF, platelet-derived growth factor (PDGF), thyrotropin releasing hormone (TRH), transforming growth factor betas (TGFβs), insulin-like growth factor (IGF-1) and the like. Differentiation can also be induced by growing cells to confluncey.

Hormones also can be advantageously used, in some embodiments, in the cell cultures for use with the methods described herein and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine and L-thyronine.

Lipids and lipid carriers also can be used, in some embodiments, to supplement cell culture media and include, but are not limited to, cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin and oleic acid unconjugated and conjugated to albumin, among others.

A population of pancreatic cells or pancreatic precursor cells generated using the methods described herein can be cultured in suspension or on a fixed substrate, or attached to a solid support, such as extracellular matrix components. For example, the pancreatic precursor cells can be grown on a hydrogel, such as a peptide hydrogel. Alternatively, the pancreatic precursor cells can be propagated on tissue culture plates or in suspension cultures. Cell suspensions can be seeded in any receptacle capable of sustaining cells, particularly culture flasks, cultures plates, or roller bottles, more particularly in small culture flasks such as, for example, 25 cm² cultures flasks. Preferably, the pancreatic precursor cells are grown on tissue culture plates. In some embodiments of the methods described herein, a population of pancreatic cells is cultured at high cell density to promote the suppression of asymmetric cell kinetics.

In some embodiments of the methods described herein, for example, nanoengineering of stem cell microenvironments can be performed. As used herein, secreted factors, stem cell-neighboring cell interactions, extracellular matrix (ECM) and mechanical properties collectively make up the “stem cell microenvironment.” Stem cell microenvironment nanoengineering can comprise the use of micro/nanopatterned surfaces, nanoparticles to control release growth factors and biochemicals, nanofibers to mimic extracellular matrix (ECM), nanoliter-scale synthesis of arrayed biomaterials, self-assembly peptide system to mimic signal clusters of stem cells, nanowires, laser fabricated nanogrooves, and nanophase thin films to expand pancreatic precursor cells.

Conditions for culturing of cells using the methods described herein should be close to physiological conditions. The pH of the culture medium should be close to physiological pH, preferably between pH 6-8, more preferably between about pH 7 to 7.8, with pH 7.4 being most preferred. Physiological temperatures range between about 30° C. to 40° C. Cells are preferably cultured at temperatures between about 32° C. to about 38° C., and more preferably between about 35° C. to about 37° C.

Cells are preferably cultured for 3-30 days according to the methods described herein, preferably at least about 7 days, more preferably at least 10 days, still more preferably at least about 14 days. Cells can be cultured, in some embodiments, substantially longer. Cells generated using the methods described herein can also be frozen using known methods such as cryopreservation, and thawed and used as needed.

Other embodiments provide for deriving clonal lines of pancreatic precursor cells by limiting dilution plating or single cell sorting. Methods for deriving clonal cell lines are well known in the art and are described for example in the Examples provided herein, as well as Puck et al., 1956; Nias et al., 1965; and Leong et al., 1985.

### Pharmacological Methods of Increasing Symmetric Division of Pancreatic Precursor Cells Cell Kinetics and Asymmetric Division

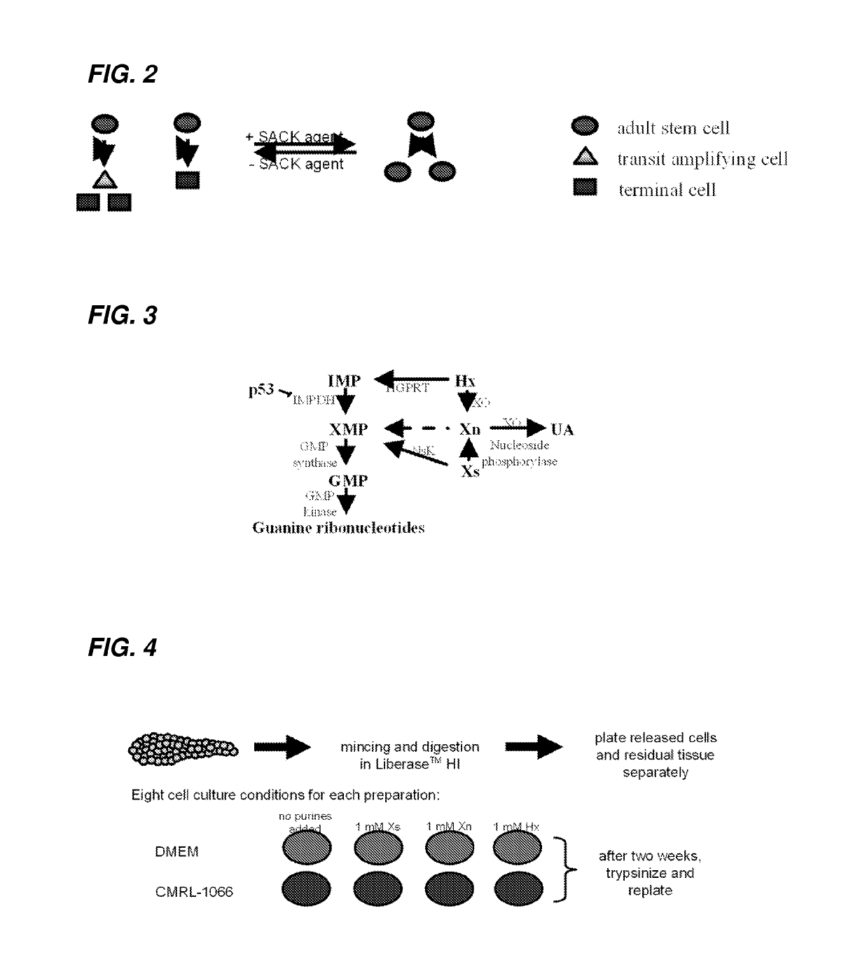

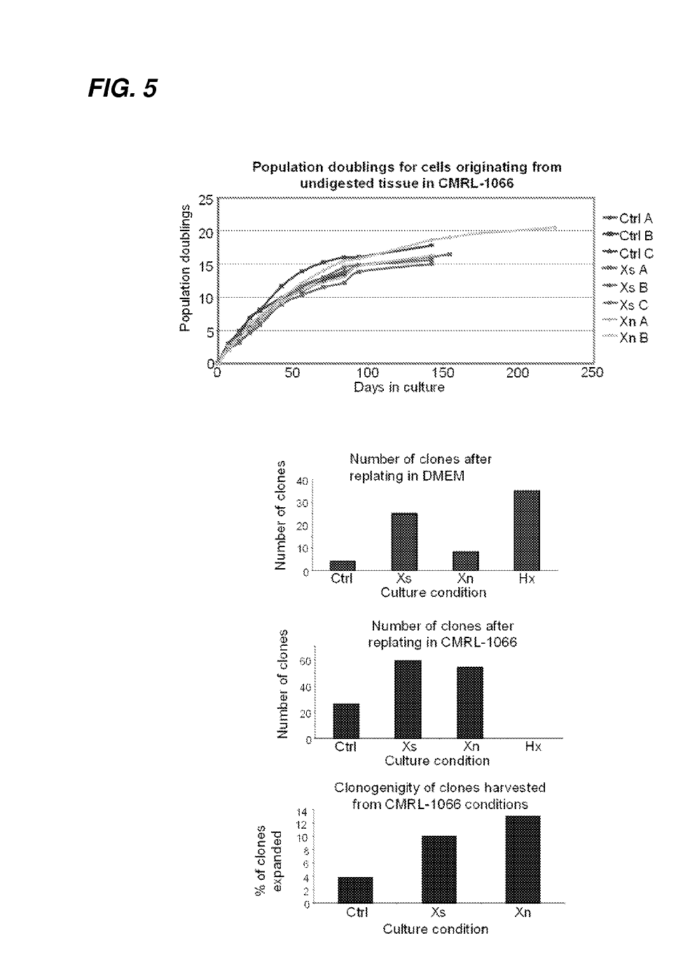

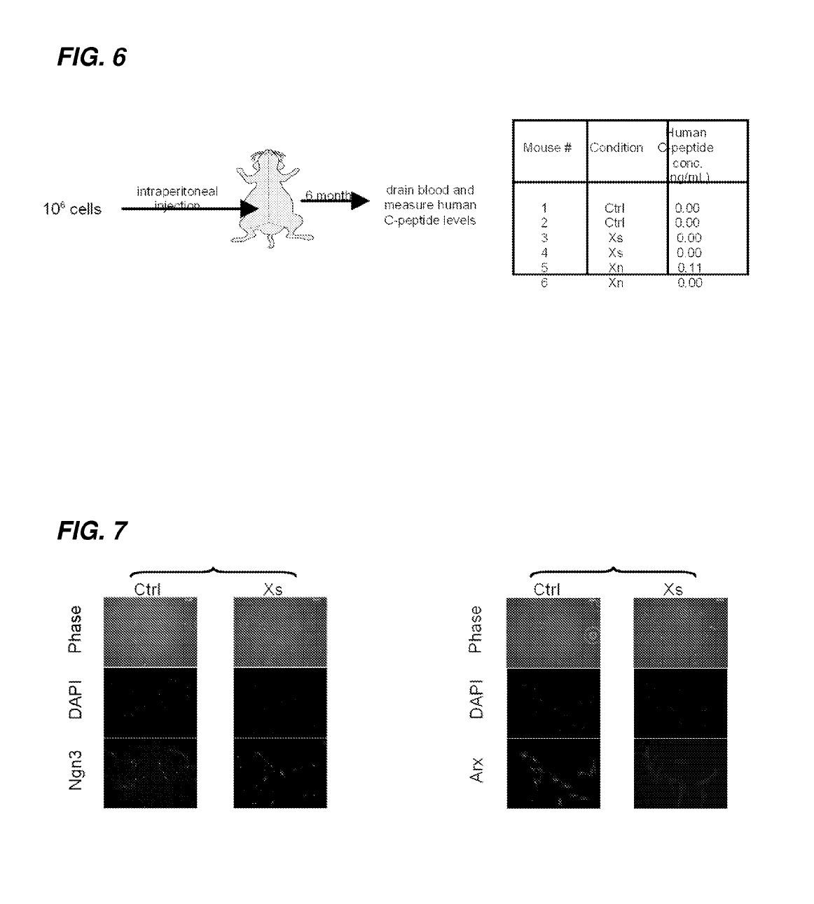

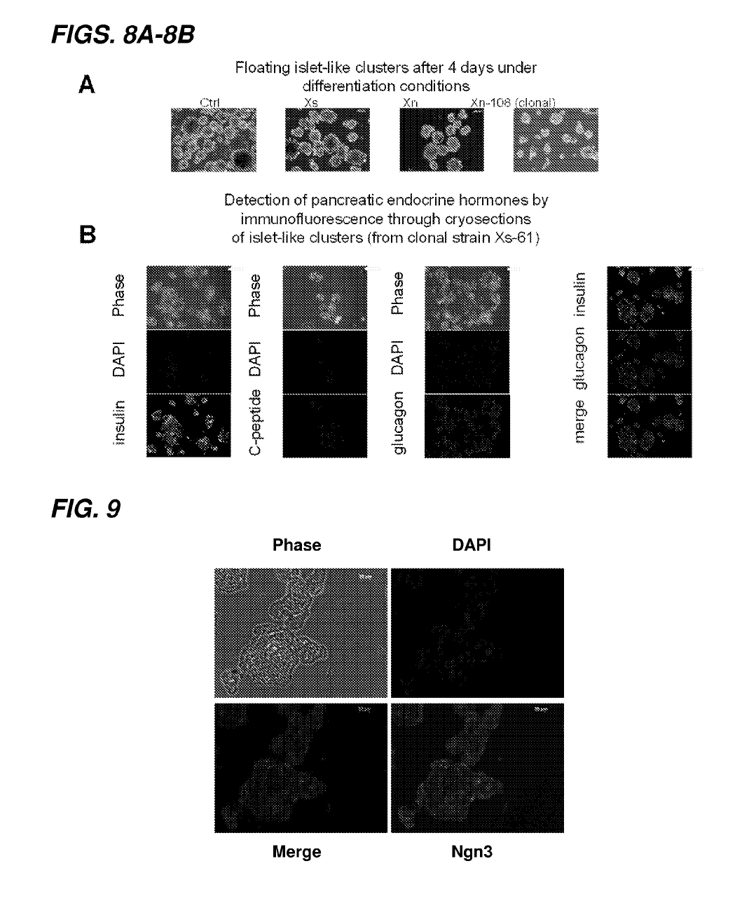

Adult somatic stem cells predominantly divide by asymmetric cell kinetics (see FIG. 2). While somatic stem or precursor cells also undergo limited symmetric divisions (that produce two identical stem cells) in developing adult tissues, such symmetric kinetics are restricted to periods of tissue expansion and tissue repair. Inappropriate symmetric somatic stem cell divisions evoke mechanisms leading to apoptosis of duplicitous stem cells (Potten and Grant, 1998). Some stem cells can also lie dormant for long periods before initiating division in response to specific developmental cues, as in reproductive tissues like the breast. However, the predominant cell kinetics state of somatic stem cells is asymmetric (Cairns, 1975; Poldosky, 1993; Loeffler and Potten, 1997).

During asymmetric cell kinetics, one daughter cell divides with the same kinetics as its stem cell parent, while the second daughter gives rise to a differentiating non-dividing cell lineage. The second daughter may differentiate immediately; or depending on the tissue, it may undergo a finite number of successive symmetric divisions to give rise to a larger pool of differentiating cells. The second daughter and its dividing progeny are called transit cells (Loeffler and Potten, 1997). Transit cell divisions ultimately result in mature, differentiated, terminally arrested cells. In tissues with high rates of cell turnover, the endpoint for differentiated terminal cells is programmed cell death by apoptosis.

Asymmetric cell kinetics evolved in vertebrates as a mechanism to ensure tissue cell renewal while maintaining a limited set of stem cells and constant adult body mass. Mutations that disrupt asymmetric cell kinetics are an absolute requirement for the formation of a clinically significant tumor mass (Cairns, 1975). In many ways, asymmetric cell kinetics provide a critical protective mechanism against the emergence of neoplastic growths that are life threatening.

In culture, continued asymmetric cell kinetics of explanted cells are a major obstacle to their expansion in vitro (FIG. 2). Ongoing asymmetric kinetics results in dilution and loss of an initial relatively fixed number of stem cells by the accumulation of much greater numbers of their terminally differentiating progeny. If a sample includes both exponentially growing cells as well as somatic stem cells, the growth of the exponentially growing cells will rapidly overwhelm the somatic stem cells, leading to their dilution.

One regulator of asymmetric cell kinetics is the p53 tumor suppressor protein. Several stable cultured murine cell lines have been derived that exhibit asymmetric cell kinetics in response to controlled expression of the wild-type murine p53. (Sherley, 1991; Sherley et al, 1995 A-B; Liu et al., 1998 A-B; Rambhatla et al., 2001). The p53 model cell lines have been used to define cellular mechanisms that regulate asymmetric cell kinetics.

In addition to p53, the rate-limiting enzyme of guanine nucleotide biosynthesis, inosine-5′-monophosphate dehydrogenase (IMPDH) is an important determinant of asymmetric cell kinetics. IMPDH catalyzes the conversion of IMP to xanthosine monophosphate (XMP) for guanine nucleotide biosynthesis. This enzymatic reaction is rate-determining for the formation of the next metabolite in the pathway, GMP, from which all other cellular guanine nucleotides are derived.

Accordingly, high levels of GNPs promote exponential kinetics, whereas low levels of GNPs promote asymmetric cell kinetics. The methods described herein provide, in part, methods for expanding pancreatic precursor cells ex vivo or in vitro by enhancing guanine nucleotide biosynthesis, thereby expanding cellular pools of GNPs and conditionally suppressing asymmetric cell kinetics.

In some embodiments of the methods described herein, expansion of human pancreatic precursor cells can start with only a single precursor cell. For example, in some embodiments, one can start with a composition or sample, such as an intact pancreatic sample, or cells isolated from such a sample, comprising or containing only 1% human pancreatic precursor cells. Using the methods described herein, these human pancreatic precursor cells can be expanded in culture, up to at least 30%, for example, at up to least 40%, up to at least 50%, up to at least 60%, up to at least 70%, up to at least 80%, up to at least 90%, up to at least 95%, up to at least 96%, up to at least 97%, up to at least 98%, up to at least 99%, or more, of the entire culture because of the suppression of asymmetric cell kinetics.

Mechanisms which function downstream of the GNPs to regulate cell kinetics (i.e. asymmetric v. symmetric) can also be used, in some embodiments of the methods described herein, to conditionally suppress asymmetric cell kinetics thereby effectively permitting a greater percent of expression by the pancreatic precursor cell. For example, in some embodiments, one can enhance expression of a protein downstream of the GNP biosynthesis pathway, if that protein inhibits asymmetric cell kinetics. Alternatively, in some embodiments, one can downregulate expression of a protein downstream of the GNP pathway if it promotes asymmetric cell kinetics.

### Pharmacological Methods for Pancreatic Precursor Cell Expansion

Somatic pancreatic precursor cells are cultivated or cultured in the presence of compounds that enhance guanine nucleotide biosynthesis in the methods described herein. This expands guanine nucleotide pools, which in turn suppress the undesired asymmetric cell kinetics, thereby permitting expansion of precursor cells resulting in production of a greater percent of pancreatic precursor cells.

Preferably, in some embodiments of the aspects described herein, the compounds are guanine nucleotide precursors (rGNPrs) or analogues or derivatives thereof. More preferably, the guanine nucleotide precursors for use in the methods described herein comprise xanthosine (Xs), xanthine (Xn), hypoxanthine (Hx), or any combination thereof. In some embodiments of the methods describe herein, the guanine nucleotide precursor is xanthosine, hypoxanthine, or a combination thereof. In some embodiments of the methods describe herein, the guanine nucleotide precursor does not comprise xanthine. In some embodiments of the methods describe herein, the guanine nucleotide precursor is xanthosine, xanthine, or a combination thereof. In some embodiments of the methods describe herein, the guanine nucleotide precursor does not comprise hypoxanthine.

The guanine nucleotide precursors (rGNPrs) or analogues or derivatives thereof can be used at effective concentrations ranging from at least 25 μM to 5000 μM. In some embodiments, the concentration of guanine nucleotide precursors (rGNPrs) or analogues or derivatives thereof ranges from 50 μM to 2000 μM. In some embodiments, the concentration of guanine nucleotide precursors (rGNPrs) or analogues or derivatives thereof is in the range of 500 μM to 1500 μM. In some embodiments, the concentration guanine nucleotide precursors (rGNPrs) or analogues or derivatives thereof used is at least 50 μM. In some embodiments, the concentration of guanine nucleotide precursors (rGNPrs) or analogues or derivatives thereof is at least 50 μM, at least 75 μM, at least 100 μM, 125 μM, at least 150 μM, at least 175 μM, at least 200 μM, 225 μM, at least 250 μM, at least 275 μM, at least 300 μM, 325 μM, at least 350 μM, at least 375 μM, at least 400 μM, 425 μM, at least 450 μM, at least 475 μM, at least 500 μM, 525 μM, at least 550 μM, at least 575 μM, at least 600 μM, 625 μM, at least 650 μM, at least 675 μM, at least 700 μM, 725 μM, at least 750 μM, at least 775 μM, at least 800 μM, 825 μM, at least 850 μM, at least 875 μM, at least 900 μM, 925 μM, at least 950 μM, at least 975 μM, at least 1000 μM, at least 1500 μM, at least 2000 μM, at least 2500 μM, at least 3000 μM, at least 3500 μM, at least 4000 μM, at least 5500 μM, at least 5000 μM, or more. One skilled in the art can determine the effective concentration necessary to suppress asymmetric kinetics of the pancreatic precursor cells to be propagated.

Cell markers useful for identifying or isolating pancreatic precursor cells generated using the methods described herein include, but are not limited to nestin, GATA-4 and HNF3, as well as markers of pancreatic beta cell fate, including insulin I, insulin II, islet amyloid polypeptide (IAPP), and the glucose transporter-2 (GLUT 2). Glucagon, a marker for the pancreatic alpha cell, can also be induced in pancreatic precursor cells generated using the methods described herein. In some embodiments of the methods described herein, a pancreatic precursor cell produces both insulin and glucagon. Expression of the pancreatic transcription factor PDX-1 can also be examined, in some embodiments.

### Uses of Expanded Pancreatic Precursor Cells

The methods described herein also provide for, in some aspects and embodiments, the administration of expanded populations of pancreatic precursor cells to a patient in need thereof, such as a patient having or predisposed to diabetes. The term “administration” as used herein refers to well recognized forms of administration, such as intravenous or injection, as well as to administration by transplantation, for example transplantation of tissue engineered islet cells or islet clusters derived from pancreatic precursor cells produced or expanded using the methods described herein. The expanded pancreatic precursor cells described herein can be used for a variety of purposes, including, but not limited, to pancreatic precursor cell therapy, such as transplantation of pancreatic precursor cells or matrices comprising such pancreatic precursor cells; tissue engineering applications, such as the use of pancreatic precursor in generation of functional artificial pancreases or functional islet cell clusters for treatment of, for example, Type I or Type II diabetes; and in gene therapy applications. The expanded pancreatic precursor cells generated using the methods described herein are also particularly useful for facilitating research on pancreatic precursor cell biology and differentiation, and for drug development and discovery.

Type I diabetes is an autoimmune disease that results in destruction of insulin-producing beta cells of the pancreas. Lack of insulin causes an increase of fasting blood glucose (around 70-120 mg/dL in nondiabetic people) that begins to appear in the urine above the renal threshold (about 190-200 mg/dl in most people). The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level 11.1 mmol/L (≧200 mg/dL).

Type 1 diabetes can be diagnosed using a variety of diagnostic tests that include, but are not limited to: (1) glycated hemoglobin (A1C) test, (2) random blood glucose test and/or (3) fasting blood glucose test, for use with embodiments of the methods described herein

The Glycated hemoglobin (A1C) test is a blood test that reflects the average blood glucose level of a subject over the preceding two to three months. The test measures the percentage of blood glucose attached to hemoglobin, which correlates with blood glucose levels (e.g., the higher the blood glucose levels, the more hemoglobin is glycosylated). An A1C level of 6.5 percent or higher on two separate tests is indicative of diabetes. A result between 6 and 6.5 percent is considered prediabetic, which indicates a high risk of developing diabetes. In some embodiments, provided herein are methods for preventing or slowing down pre-diabetes from escalating into diabetes using the pancreatic precursor cells generated using the methods described herein. Therefore, methods for prevention of diabetes are also provided herein.

As used herein, the term “HBA1c” or “A1C” refers to glycosylated hemoglobin or glycated hemoglobin, and is an indicator of blood glucose levels over a period of time (e.g., 2-3 months). The level of HBA1c is “reduced” if there is a decrease of at least 20%, at least 30%, at least 40%, at least 50%, at least 60%, at least 70%, at least 80%, at least 90%, at least 95%, or more upon treatment with pancreatic precursor cells generated using the methods described herein compared to the level of HBA1c prior to the onset of treatment in the subject. Similarly, ketone bodies are “reduced” if there is a decrease of at least 20%, at least 30%, at least 40%, at least 50%, at least 60%, at least 70%, at least 80%, at least 90%, at least 95%, or more upon treatment with differentiated islets treated with pancreatic precursor cells generated using the methods described herein.

The Random Blood Glucose Test comprises obtaining a blood sample at a random time point from a subject suspected of having diabetes. Blood glucose values can be expressed in milligrams per deciliter (mg/dL) or millimoles per liter (mmol/L). A random blood glucose level of 200 mg/dL (11.1 mmol/L) or higher indicates the subject likely has diabetes, especially when coupled with any of the signs and symptoms of diabetes, such as frequent urination and extreme thirst.

For the fasting blood glucose test, a blood sample is obtained after an overnight fast. A fasting blood glucose level less than 100 mg/dL (5.6 mmol/L) is considered normal. A fasting blood glucose level from 100 to 125 mg/dL (5.6 to 6.9 mmol/L) is considered prediabetic, while a level of 126 mg/dL (7 mmol/L) or higher on two separate tests is indicative of diabetes.

Type 1 diabetes can also be distinguished from type 2 diabetes using a C-peptide assay, which is a measure of endogenous insulin production. The presence of anti-islet antibodies (to Glutamic Acid Decarboxylase, Insulinoma Associated Peptide-2 or insulin), or lack of insulin resistance, determined by a glucose tolerance test, is also indicative of type 1, as many type 2 diabetics continue to produce insulin internally, and all have some degree of insulin resistance.

In some embodiments, the treatment methods further comprise selection of a subject to be administered the cells produced using the methods described herein. As used herein, the term “selecting a subject having diabetes” refers to the diagnosis of a subject with diabetes prior to the onset of treatment of the subject with or transplantation to the subject of, for example, a population of pancreatic precursor cells or a population of differentiated islet cells. Diabetes can be diagnosed, for example, using a glycosylated hemoglobin (A1C) test, a random blood glucose test and/or a fasting blood glucose test, as described herein

### Differentiating Islet Cell and Monitoring Differentiation

The expanded pancreatic precursor cells generated using the methods described herein can be subsequently differentiated into islet cells for treatment and transplantation methods.

Accordingly, in some embodiments, the methods further comprise monitoring islet cell differentiation. Such monitoring of islet cell differentiation includes measuring the emergence of cell surface markers specific for differentiated islets, and/or measuring insulin production, glucagon production, somatostatin production, or any combination thereof. In some embodiments of the methods, the islet cells produced by differentiation of the pancreatic precursor cells generated using the methods described herein produce or secrete insulin or glucagon, but not both.

In some embodiments of the methods described herein, the pancreatic precursor cells can be induced to differentiate following expansion in vitro, prior to administration to the individual. Preferably, the pool of guanine ribonucleotides is decreased at the same time differentiation is induced, for example by removal of the rGNPr from the culture medium (if a pharmacological approach has been used) or by downregulating expression of the transgene.

Other substrates can be used to induce differentiation such as collagen, fibronectin, laminin, MATRIGEL™ (Collaborative Research), and the like. Differentiation can also be induced by leaving the cells in suspension in the presence of a proliferation-inducing growth factor, without re-initiation of proliferation.

Differentiation can be determined using immunocytochemistry techniques well known in the art. Immunocytochemistry (e.g. dual-label immunofluorescence and immunoperoxidase methods) utilizes antibodies that detect cell proteins to distinguish the cellular characteristics or phenotypic properties of differentiated cell types compared to markers present on pancreatic precursor cells.

Techniques for detecting insulin or glucagon include, for example, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al., J. Biol. Chem. 255:4980-4983 (1980)); radioimmunoassays (RIA); enzyme-linked immunosorbent assays (ELISA) as described, for example, by Raines et al., J. Biol. Chem. 257:5154-5160 (1982); immunocytochemical techniques, including the use of fluorochromes (Brooks et al., Clin. Exp. Immunol. 39:477 (1980)); and neutralization of activity (Bowen-Pope et al., Proc. Natl. Acad. Sci. USA 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

The differentiated state of cells can also be detected by measuring the rate of insulin or glucagon production. The differentiated state of cells can also be detected by analyzing the cell surface markers on the cells. For example, differentiated islet β-cells express PDX-1, but not CK-19 (see, e.g., Beattie et al. (1999) Diabetes 48:1013). Techniques for detecting cell surface markers are well known in the art and are described in, e.g., Harlow and Lane, USING ANTIBODIES (1999).

The differentiated state of cells can also be detected by analyzing the expression levels of various proteins by the cell. Methods of detecting protein expression are well known in the art and are described in, e.g., Ausubel et al., supra.

### Administering Pancreatic Cells to a Subject

Cultured pancreatic precursor cells or differentiated pancreatic precursor cells generated using the methods described herein can be administered to a subject by any means known to those of skill in the art. Suitable means of administration include, for example, intravenous, subcutaneous, via the liver portal vein, by implantation under the kidney capsule, or into the pancreatic parenchyma. After generating the pancreatic precursor cells, the cells can be administered after a period of time sufficient to allow them to convert from asymmetric cell kinetics to exponential kinetics, typically after they have been cultured from 1 day to over a year. In some embodiments, the pancreatic precursor cells are cultured for at least 3-30 days, at least 4-14 days, or at least 7 days.

Between 10⁴ and 10¹³ pancreatic precursor cells or differentiated pancreatic precursor cells generated using the methods described herein per 100 kg person are administered per infusion. Preferably, between about 1-5×10⁴ and 1-5×10⁷ cells are infused intravenously per 100 kg person. More preferably, between about 1×10⁴ and 5×10⁶ cells are infused intravenously per 100 kg person.

In some embodiments of the methods, a single administration of cells is provided. In other embodiments of the methods, multiple administrations are used. Multiple administrations can be provided over periodic time periods such as an initial treatment regime of 3-7 consecutive days, and then repeated at other times.

Additional exemplary methods of administering pancreatic precursor cells or differentiated islet cells to a subject, particularly a human subject, include injection or transplantation of the cells into target sites in the subject. The pancreatic precursor cells or differentiated islets can be inserted into a delivery device which facilitates introduction, by injection or transplantation, of the cells into the subject. Such delivery devices include tubes, e.g., catheters, for injecting cells and fluids into the body or particular organ (e.g., pancreas) of a recipient subject. In some preferred embodiments, the tubes additionally have a needle, e.g., a syringe, through which the cells described herein can be introduced into the subject at a desired location. The pancreatic precursor cells or differentiated islets can be inserted into such a delivery device, e.g., a syringe, in different forms. For example, the cells can be suspended in a solution, or alternatively embedded in a support matrix when contained in such a delivery device.

In some embodiments of the methods, cultured pancreatic precursor cells or differentiated islet cells in an intact matrix can be administered to a subject. Support matrices in which the pancreatic precursor cells or differentiated islet cells can be incorporated or embedded include matrices that are recipient-compatible and that degrade into products that are not harmful to the subject. The support matrices can be natural (e.g. collagen etc.) and/or synthetic biodegradable matrices. Synthetic biodegradable matrices include synthetic polymers such as polyanhydrides, polyorthoesters, and polylactic acid; see also, for example, U.S. Pat. No. 4,298,002 and U.S. Pat. No. 5,308,701. Alternatively, the matrix can be disrupted by a protease before the pancreatic precursor cells or differentiated islet cells are administered to the subject. Suitable proteases for disrupting the matrix include, for example, streptokinase or tissue plasminogen activator.

Cells can be extracted from the subject to be treated, i.e., autologous, (thereby avoiding immune-based rejection of the implant) or can be from a second subject, typically an HLA compatible or surface marker/immunologically compatible donor i.e., heterologous. In either case, administration of pancreatic precursor cells or differentiated islet cells can be combined with an appropriate immunosuppressive treatment.

The pancreatic precursor cells or differentiated islet cells can be in formulations suitable for administration, such as, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. As used herein, the term “solution” includes a pharmaceutically acceptable carrier or diluent in which the progenitor cells or differentiated islets remain viable. Pharmaceutically acceptable carriers and diluents include saline, aqueous buffer solutions, solvents and/or dispersion media. The use of such carriers and diluents is known in the art. The solution is preferably sterile and fluid to the extent that easy syringability exists.

Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions comprising the cells generated using the methods described herein can be prepared by incorporating cells as described herein in a pharmaceutically acceptable carrier or diluent and, as required, other ingredients enumerated above, followed by filtered sterilization. In the practice of the methods described herein, such compositions can be administered, for example, by direct surgical transplantation under the kidney, intraportal administration, intravenous infusion, or intraperitoneal infusion.

Injection solutions and suspensions can be prepared from sterile powders, granules, and tablets. The dose administered to a patient, in the context of the present invention should be sufficient to effect a beneficial therapeutic response in the patient over time. The dose will be determined by the efficacy of the particular cells employed and the condition of the patient, as well as the body weight or surface area of the patient to be treated. The size of the dose also will be determined by the existence, nature, and extent of any adverse side-effects that accompany the administration of a particular cell type in a particular patient.

In determining the effective amount of the cells, for example, pancreatic precursor cells or differentiated islet cells, to be administered in the treatment or prophylaxis of conditions owing to diminished or aberrant insulin expression, the physician evaluates cell toxicity, transplantation reactions, progression of the disease, and the production of anti-cell antibodies. For administration, cells generated using various embodiments of the methods described herein can be administered in an amount effective to provide normalized glucose responsive-insulin production and normalized glucose levels to the subject, taking into account the side-effects of the cell type at various concentrations, as applied to the mass and overall health of the patient. Administration can be accomplished via single or divided doses.

### Monitoring Efficacy of Treatment

The efficacy of a given treatment for diabetes can be determined by the skilled clinician. However, a treatment is considered “effective treatment,” as the term is used herein, if any one or all of the signs or symptoms of diabetes, e.g., Type 1 diabetes, for example, hyperglycemia are altered in a beneficial manner, other clinically accepted symptoms or markers of disease are improved, or ameliorated. In some embodiments, the improvement is seen as a need for fewer insulin injections, less insulin, fewer episodes of hospitalization, and/or longer intervals between hospitalizations, than the individual has experienced prior to treatment with the pancreatic precursor cells or pancreatic islet cells generated using the methods as described herein. Efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing loss of insulin production; or (2) relieving the disease, e.g., causing regression of symptoms, increasing insulin or glucagon production. The methods can also be used, in some embodiments, to prevent or reduce the likelihood of the development of a complication of Type 1 diabetes, e.g., diabetic retinopathy.

An effective amount for the treatment of diabetes means that amount which, when administered to a mammal in need thereof, is sufficient to result in effective treatment as that term is defined herein. Efficacy of a treatment can be determined by assessing physical indicators of, for example, Type 1 diabetes, such as hyperglycemia, normoglycemia, ketone bodies, hypoinsulinemia, etc.

### Gene Therapy Applications

In some embodiments, the pancreatic precursor cells generated using the methods described herein can be further genetically altered prior to reintroducing the cells into the individual for gene therapy, to introduce a gene whose expression has therapeutic effect on the individual.

For example, in some embodiments, the pancreatic precursor cells may have a defective gene that inhibits insulin production. By introducing normal genes in expressible form, individuals suffering from such a deficiency can be provided the means to compensate for genetic defects and eliminate, alleviate or reduce some or all of the symptoms of the deficiency.

A vector can be used for expression of the transgene encoding a desired wild type hormone or a gene encoding a desired mutant hormone. Preferably, as described above, the transgene is operably linked to regulatory sequences required to achieve expression of the gene in, for example, the pancreatic precursor cells or the cells that arise from the pancreatic precursor cells after they are infused into an individual. Such regulatory sequences include a promoter and a polyadenylation signal. The vector can contain any additional features compatible with expression in pancreatic precursor cells or their progeny, including, for example, selectable markers.

### Screening Candidate Agents for Islet Cell Differentiation

The pancreatic precursor cells or differentiated islet-cells generated using the methods described herein are also useful, in some aspects and embodiments, for drug screening applications. Candidate agents to be screened for islet differentiation properties can be contacted with the cells in a conditioning phase, or alternatively can be embedded in a matrix. In addition, a candidate agent can be supplied in a cell culture medium or as a supplement, in some embodiments. A candidate agent is determined to enhance islet cell differentiation if there is an increase in the number of clusters or cluster size (e.g., area) compared to a control culture of the same cells in the absence of the candidate agent, or if the clusters form within a shorter time period compared to formation of clusters without a candidate agent. In other embodiments, a candidate agent is determined to enhance islet cell differentiation if there is an increase in insulin or glucagon production by the islet cells.

Any agent can be tested using the above-described cell culture system including e.g., small molecules, proteins, peptides, nucleic acids, drugs, among others. It is contemplated herein that different doses of each candidate agent are tested using the above-described system.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Small molecule libraries can be obtained commercially and screened for islet cell differentiation efficacy by one of skill in the art.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Materials and Methods

Most methods currently used to isolate and culture human pancreatic beta-cell precursors rely on the separation of pancreatic islets prior to cell culture. In contrast, the methods described herein utilize all the cells and tissue cell fragments obtained after enzymatic digestion of intact human pancreatic tissue, and culture these cells and tissue cell fragments in medium supplemented with guanine ribonucleotide precursors (GrNPs) to produce human pancreatic stem cells that are precursors for human pancreatic islet cells.

More specifically, after mincing of intact pancreatic tissue (harvested by the National Disease Research Interchange from donors within 4 hours of death), the pancreatic tissue was digested with liberase H (Roche). Two successive digestions of 20 and 30 minutes were performed at 37° C. in serum-free Dulbecco's modified Eagle's medium (DMEM) containing 1 mg/mL LIBERASE H. The cells and cell clusters released after each of these digestions were cultured.

In one embodiment of the methods described herein, termed herein as “Method 1,” twice digested pancreas digestion products can be cultured in DMEM supplemented with 10% dialyzed fetal bovine serum (DFBS). In one such example, pancreas digestion products cultured in DFBS were further subdivided equally among the following four medium supplementation conditions: no GrNPs added (“control”), 1 mM xanthosine (Xs), 1 mM xanthine (Xn), or 1 mM hypoxanthine (Hx).

In another embodiment of the methods described herein, termed herein as “Method 2,” the same two successive digestion procedure can be used, but the final tissue fragments remaining after the second digestion can also be cultured independently. For this embodiment of the methods, CMRL-1066 medium can be used as a second base culture medium in parallel with DMEM for all cultured pancreatic cell and tissue preparations. In one such example of Method 2, CMRL-1066 medium was supplemented with 10% DFBS and 2 mM L-glutamine and subdivided for the same GrNP supplementations as indicated for DMEM-10% DFBS medium in Method 1.

Using both methods and under all the conditions tested, cells adhered to culture dishes and were allowed to grow for two weeks. However, when the cultured attached cells were subsequently trypsinized and passaged to a new dish, only a subset of conditions was permissive for cell survival and continued proliferation. For cells cultured using the embodiment of the methods described herein termed Method 1, only cells originating from the second digestion and grown in DMEM-10% DFBS supplemented with GrNPs proliferated sufficiently to form clones (See Table 1).

For cells cultured using the embodiment of the methods described herein termed Method 2, similar cultures developed in DMEM-10% DFBS and yielded a similar outcome, with the exception being that several clones grew in the control (GrNP-free) condition (See Table 1). In both of these embodiments of the methods, supplementation with Xs or Hx was more effective in enhancing clonal cell proliferation than Xn, whose effect was not statistically different than control GrNP-free culture conditions. Accordingly, in some embodiments of the methods described herein, the culture medium comprise Xs, Hx, or a combination thereof. In some embodiments, the culture medium does not

TABLE 1
Cell clones developed from second digestion of pancreas tissue in
Method 1 and Method 2 cultures developed with DMEM-10%
DFBS + GrNP supplementation

#clones after
1 passage

| Supplement | Method 1 | Method 2 | Poisson p¹ |
|---|---|---|---|
| Control | 0 | 4 | — |
| 1 mM Xs | 13 | 12 | <2 × 10⁻⁷ |
| 1 mM Xn | 3 | 5 | 0.09 |
| 1 mM Hx | 22 | 13 | <6 × 10⁻¹² |

¹Based on averages of Method 1 and Method 2 values.

comprise Xn.

For CMRL-1066-10% DFBS cultures developed using the embodiment of the methods described herein as Method 2, only cells derived from the final digested tissue fragments proliferated sufficiently to form clones. The number of clones obtained in the Xn- or Xs-supplemented cultures was approximately 50% greater than the number obtained in control (See Table 2). However, Hx-supplemented cultures did not produce any clones. Accordingly, in some embodiments of the methods described herein, the culture medium comprise Xn, Xs, or a combination thereof. In some embodiments, the culture medium does not comprise Hx.

The proportion of clones developed from tissue fragments cultured in CMRL-1066 medium that could be further expanded to full cultures was also increased 2.6-fold and 3.4-fold by Xs and Xn, respectively (See Table 2). Cell clone colonies were also isolated and expanded from Hx supplemented cultures developed by Method 1 (Table 1, 1 mM Hx).

After isolating clonal cell colonies produced by the embodiment of the methods described herein termed Method 2 (Table 2) for expansion, the remaining unselected cell clones were pooled and expanded as well (i.e., polyclonal strains). Population doubling analyses were performed based on the passage of the expansion pool cultures. Only polyclonal strains grown in Xn-supplemented medium proliferated for 20 population doublings (corresponding to approximately a 106-fold cell expansion).

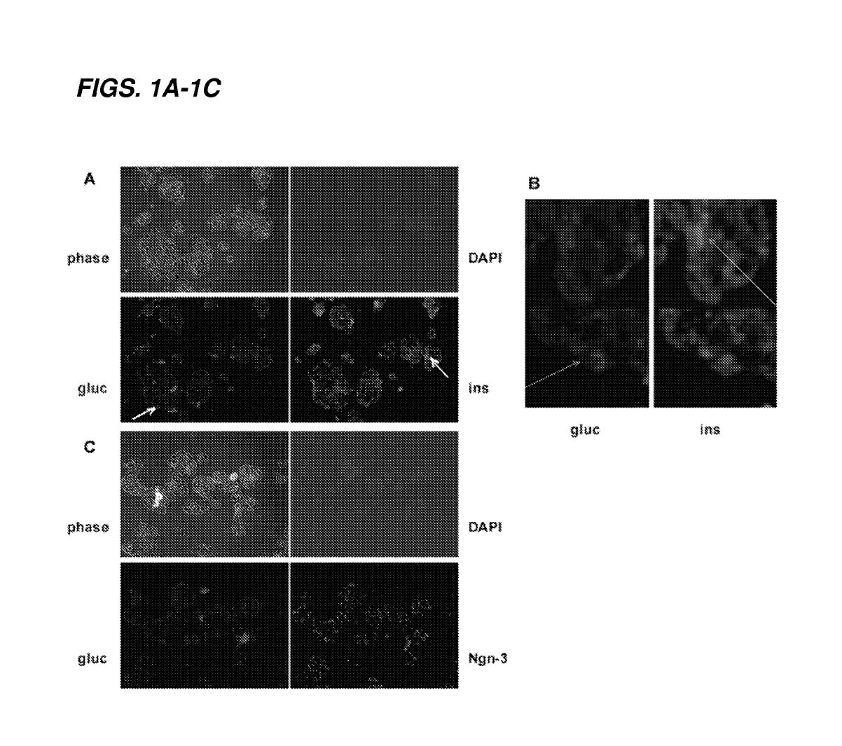

Under GrNP-supplementation conditions for active proliferation, expanded polyclonal and clonal cell strains generated using the methods described herein express the intermediary filament vimentin, which identifies them as mesenchymal type cells. They also express neurogenin-3 (Ngn-3), which signifies a neuroendocrine progenitor phenotype. However, when cultured under GrNP-free conditions for pancreatic islet differentiation (CMRL-1066 medium supplemented with 1% fatty acid-free bovine serum albumin, 2 mM L-glutamine, and 1× insulin-transferrin-selenium), they continue to express the endocrine progenitor marker Ngn-3, but now many cells also co-express the pancreatic islet hormones insulin and glucagon (See FIGS. 1A-1C, for example). Cells are also detected that express only insulin or glucagon, but not both (FIGS. 1A and 1B). The single-positive cells indicate continued development of mature beta-cell and alpha-cell phenotypes. The co-expressing cells are predicted to be, without wishing to be bound or limited by theory, common progenitors that give rise to beta-cells (insulin-producing) and alpha cells (glucagon-producing). These in vitro properties establish the cell strains produced by the embodiments of the methods described herein termed Method 1 and Method 2 as pancreatic stem cells that have the capacity to produce progeny cells with mature pancreatic islet

TABLE 2
Cell clones developed from twice-digested fragments of pancreas
tissue in Method 2 cultures developed with CMRL-1066-10%
DFBS + GrNP supplementation

| | #clones after 1 passage | | |
|---|---|---|---|
| Supplement | Method 2 | Poisson p¹ | Clone expansion rate² |
| Control | 26 | — | 3.8% |
| 1 mM Xs | 59 | 1 × 10⁻⁸ | 10% |
| 1 mM Xn | 54 | <6 × 10⁻⁷ | 13% |
| 1 mM Hx | 0 | 5 × 10⁻¹² | N/A |

¹Based on averages of Method 1 and Method 2 values.
²Percent of isolated clones that could be expanded to full cultures

function.

### Methods for Production of Adult Pancreatic Stem Cell Strains.

Tissue-specific stem cells are notoriously difficult to identify, isolate, and expand in culture. As described herein, we have recently solved the tissue-specific stem cell expansion problem by developing a general method for selective expansion and long-term propagation of diverse tissue stem cells in culture. Thus far, the method has proven effective for expansion of adult rat hepatocytic stem cells and adult rat cholangiocytic stem cells (Lee et al., 2003); skeletal muscle stem cells; and hair follicle stem cells (Sherley and King, 2010; Huh et al., submitted); and human adult hepatic stem cells (Sherley and Panchalingam, 2010). Described herein are novel methods and results for adult mouse pancreatic precursor cells and adult human pancreatic precursor cells.

The employed tissue-specific stem cell expansion technology is based on the long-held concept that individual tissue-specific stem cells undergo asymmetric self-renewal (Potten and Morris, 1988; Loeffler and Potten, 1997; Sherley, 2002, 2005). Asymmetric self-renewal is defined by stem cell divisions that yield a new stem cell and its sister, which becomes the progenitor for differentiated cells in the tissue. We were the first to show that individual mammalian cells can undergo asymmetric self-renewal (Sherley et al., 1995; Rambhatla et al., 2001, 2005). Thereafter, others (Huang et al., 1999) and the inventors (Lee et al., 2003) showed that ex vivo-derived human and rat tissue-specific stem cells, respectively, had this property that was first postulated nearly 30 years ago (Lajtha, 1979).

The asymmetric self-renewal of tissue-specific stem cells poses a major barrier to their expansion in culture (Sherley, 2002; Lee et al., 2003; Pare and Sherley, 2006). In tissues, asymmetric self-renewal maintains a constant fraction of stem cells. In culture, because terminally differentiated progeny cells are not removed as in tissues, they accumulate. The accumulation of progeny cells causes the loss of asymmetrically self-renewing stem cells simply by dilution. Using engineered cell lines that modeled asymmetric self-renewal, the inventors discovered a p53-dependent pathway that controls the self-renewal pattern of tissue-specific stem cells. This pathway makes it possible to reversibly shift tissue-specific stem cells from asymmetric self-renewal to symmetric self-renewal. Symmetric self-renewal divisions produce two stem cells, leading to their exponential expansion and limiting the production of diluting differentiating progeny cells. Hence, the method is termed herein as “SACK” for “suppression of asymmetric cell kinetics” (Lee et al., 2003; Pare and Sherley, 2006; Sherley et al., 2010; Sherley and King, 2010; Sherley and Panchalingam, 2010; Huh et al., submitted). SACK promotes the selective expansion of any tissue-specific stem cell type from any mammalian species because of the universality of asymmetric self-renewal by stem cells in mammalian tissue plans. The method is selective, because it can be applied under conditions that arrest the division of differentiating progeny cells, but not symmetrically cycling stem cells.

FIG. 3 illustrates the underlying molecular and biochemical pathway responsible for the SACK effect. Asymmetric cell kinetics requires the repression of the expression of type II inosine monophosphate dehydrogenase (IMPDH II) by the p53 tumor suppressor protein. IMPDH II catalyzes the rate-limiting step for guanine ribonucleotide (rGNP) biosynthesis. Several xanthine nucleus compounds have been defined as SACK agents (Lee et al., 2003; Sherley and King, 2010) due to their ability to promote rGNP production despite p53′s down-regulation of IMPDH II. By circumventing the p53/IMPDH II-dependent regulation of rGNP pools, SACK agents shift explanted adult tissue stem cells from asymmetric cell kinetics to symmetric cell kinetics, and thereby promote their exponential expansion (Lee et al., 2003; Rambhatla et al., 2005; Pare and Sherley, 2006). Three purine nucleotide precursors have been shown to have this ability due to their entry points into the rGNP biosynthetic pathway (FIG. 2).

Hypoxanthine (Hx) is postulated to increase flux through the regulated pathway by increasing the level of IMP, the IMPDH substrate. Xanthine (Xn) and xanthosine (Xs) are postulated to bypass the point of p53 regulation altogether by promoting formation of XMP, the product of the IMPDH II reaction. Consistent with their predicted effects on p53-induced asymmetric cell kinetics, all three compounds have been shown to induce reversible shifts from asymmetric cell kinetics to symmetric cell kinetics (Sherley, 1991; Sherley et al., 1995; Liu et al., 1998; Lee et al., 2003; Sherley and King, 2010; Huh et al., submitted). When tissue cell preparations are cultured in the presence of one or more of these compounds, tissue-specific stem cells selectively multiply exponentially as a result of the SACK-induced reversible shift from asymmetric cell kinetics to symmetric cell kinetics. The resulting tissue stem cell strains have a greatly reduced frequency of cell variants with stably disrupted asymmetric cell kinetics (e.g., p53 mutant cells), because exponential SACK expansion nullifies the growth advantage of cells that acquire growth-activating mutations (Lee et al., 2003).

Provided herein are novel methods of producing and propagating adult human pancreatic stem cells based, in part, on SACK expansion. For these methods, transgenic XPRT was neither required nor investigated. Rather, purines were supplemented into the method developed by Gershengorn and others for expanding human pancreatic mesenchymal cells with the potential to differentiate into clusters of insulin-secreting cells (Gershengorn et al., 2004; Davani et al., 2007). The reported success of the Gershengorn method has been questioned by others (Kayali et al., 2007); and in side-by-side trials versus an epithelial cell method, neither method avoided the well known loss of insulin-producing capability of pancreatic cell cultures after their expansion to the extent required to support transplantation therapy development (Kayali et al., 2007).

However, in complete contrast to previously described methods, the methods of producing and propagating adult human pancreatic stem cells described herein start by dissociating the entire pancreas of donors, instead of starting with isolated islets. Unexpectedly, despite using whole pancreatic samples, as opposed to the isolated islet cells, as standard in the art, we found that SACK agents enhanced all aspects of our modified Gershengorn method. The number of initial cell colonies produced by the methods described herein increased by approximately 50% and the ability to propagate initial colonies as clonal strains increased 3-fold, relative to the standard methods in the art; and only SACK agent-supplemented cultures prepared using the methods described herein exceeded 20 population doublings.

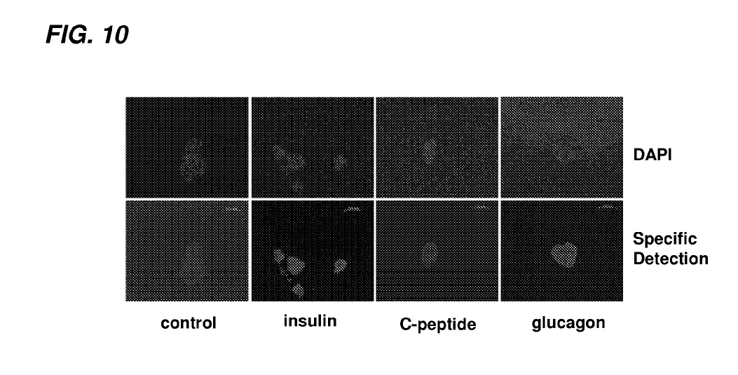

As shown herein, under serum-free and SACK-free conditions, the expanded cells form islet-like clusters that show nuclear expression of the endocrine progenitor cell marker Neurogenin 3 (FIG. 9), indicative of their potential to produce pancreatic endocrine cells, including insulin-secreting beta-cells. Further, FIG. 10 provides exemplary data that demonstrate that differentiated cell clusters produced from SACK-expanded, adult human pancreatic stem cells produced using the methods described herein have properties of functional islets. They produce insulin, C-peptide (a physiological byproduct of insulin secretion), and glucagon.

1. A method of culturing and expanding somatic pancreatic precursor cells in vitro, comprising:
a) twice digesting a population of pancreatic cells isolated from an intact pancreatic tissue sample obtained from a mammal, wherein said population of pancreatic cells comprises pancreatic alpha cells, pancreatic beta cells, pancreatic delta cells, pancreatic polypeptide (PP) cells, acinar cells, ductal cells, pancreatic precursor cells, mesenchymal cells, fibroblasts, or a mixture or combination thereof;
b) culturing said population of pancreatic cells in a culture medium that permits cell growth under conditions, and for a time sufficient, to permit cell growth, wherein the culture medium comprises at least 50 μM of a guanine nucleotide precursor selected from xanthosine, xanthine, or hypoxanthine, or an analogue or derivative thereof, wherein said guanine nucleotide precursor, analogue or derivative thereof suppresses asymmetric cell kinetics, thereby allowing exponential growth and expansion of said pancreatic precursor cells.

2. The method of claim 1, wherein said twice digesting step does not further comprise purifying, enriching for, or separating pancreatic alpha cells, pancreatic beta cells, or a combination thereof, prior to the step of culturing.

3. The method of claim 1, wherein said guanine nucleotide precursor is a combination of xanthosine and hypoxanthine.

4. The method of claim 1, wherein said guanine nucleotide precursor does not comprise xanthine.

5. The method of claim 1, wherein said guanine nucleotide precursor is a combination of xanthosine and xanthine.

6. The method of claim 1, wherein said guanine nucleotide precursor does not comprise hypoxanthine.

7. The method of claim 1, wherein said guanine nucleotide precursor is present in an amount of at least 200 μM.

8. The method of claim 7, wherein said guanine nucleotide precursor is present in an amount of 200 μM-2 mM.

9. The method of claim 7, wherein said guanine nucleotide precursor is present in an amount of 500 μM-2 mM.

10. The method of claim 1, wherein said pancreatic precursor cells can differentiate into a pancreatic alpha cell or a pancreatic beta cell.

11. The method of claim 1, wherein said pancreatic precursor cells produce insulin, glucagon, or a combination thereof.
